(19) United States
(12) Patent Application Publication
(10) Pub. No.: US 2007/0083169 A1
(43) Pub. Date: Apr. 12, 2007

(54) Towelette

(76) Inventor: Philip Krugman, Tampa, FL (US)

Correspondence Address: KEVIN J. MCNEELY, ESQ., 5335 WISCONSON AVENUE, NW, SUITE 440, WASHINGTON, DC, 20015, US

(21) Appl. No.: 11/537,690

(22) Filed: Oct. 2, 2006

Related U.S. Application Data
(60) Provisional application No. 60/725,617, filed on Oct. 12, 2005.

(51) Int. Cl. A61F 13/00

(52) U.S. Cl. 604/304; 424/443

(57) ABSTRACT

A body piercing towelette includes a composition of water and salt applied to a fibrous sheet. The water may undergo further processing such as, deonization, filtering or sterilization. The salt may be a sea salt, such as, for example, coral reef sea salt.

### CROSS-REFERENCE TO RELATED APPLICATION

This utility patent application claims priority to U.S. provisional patent application 60/725,617 filed on Oct. 12, 2005.

### TECHNICAL FIELD

The invention is related to towelettes, and more particularly, to towelettes used to cleanse and heal pierced skin and/or recent tattoo areas on a person's body.

### BACKGROUND

Body piercings and tattoos typically require care to keep the area clean and moist and to promote healing. Typically, bottled solutions or creams are available to apply to the skin. However, once the bottle is opened it may be exposed to oxidation, outside elements and potential contamination. Thus, a need exists to improve the sterility and freshness of tattoo and piercing aftercare products.

### SUMMARY

In one general aspect, a body piercing towelette includes a composition of water and salt applied to a fibrous sheet. Embodiments may include one or more of the following features. For example, the water may undergo further processing such as, deonization, filtering or sterilization. The salt may be a sea salt, such as, for example, coral reef sea salt.

The composition may include additional ingredients, such as, for example, a preservative such as lysozyme chloride or an anti-inflammatory agent such as yucca glauca root extract. Other ingredients may include anti-irritant and anti-hemorrhaging agents and antioxidant such as Vitamin C.

The towelette may be enclosed in a sealed container, such as, for example, a moisture proof foil wrapper.

In another general aspect, a method of manufacturing a body piercing towelette includes mixing a composition of water and sea salt, applying the composition to a fibrous sheet and enclosing the fibrous sheet in a sealed container.

In a further general aspect, a method of treating a pierced area of a person's skin includes removing a towelette impregnated with a mixture of sea salt and water from a sealed foil wrapper and swabbing the pierced area of the skin with the towelette.

### DESCRIPTION OF THE DRAWING

FIG. 1 shows a method of preparing a towlette.

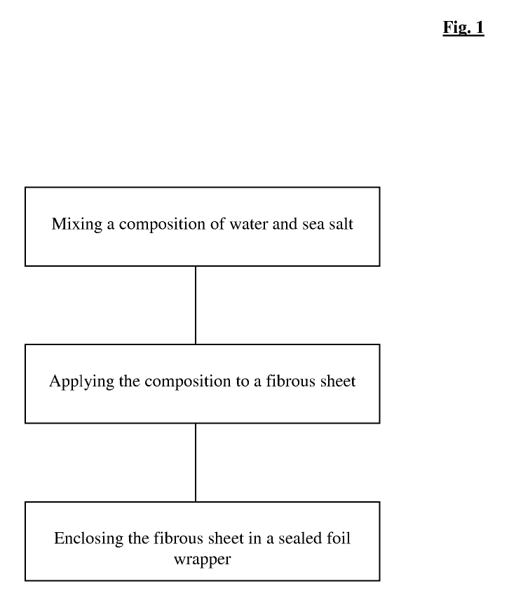

### DETAILED DESCRIPTION

The invention relates to a composition impregnated in a towelette cloth for use on/in a person's skin for cleansing and healing a pierced area or recent tattoo area of a person's skin. The towelette containing the mixture is encased in a leakproof package, typically with a foil lining or in a waterproof container to hold multiple towelettes. The composition includes a saline solution, such as, for example, a mixture of sea salt or coral reef sea sale and sterile, filtered or deonized water. Sea salts contain minerals—such as calcium, potassium, magnesium, sodium, iron, sulphur, phosphorus and chlorine that have a rejuvenating and revitalizing effect on skin. Benefits may include deep pore cleansing, removal of rough, dry or scarred skin, helping to purify and slough off dead skin cells, promoting the removal of toxins from the skin and guarding against moisture loss.

The composition may also include other ingredients, such as, for example, emollients, moisturizers, emollients, humectants, disinfectants, anti-inflammatory agents, antioxidants, anti-hemorrhaging agents, cleansers, restoratives and preservatives. For example, the anti-inflammatory agents may include yucca glauca extract and the preservative may be cetyl pyridinium chloride or lysozyme chloride. Moisturizers may include wheatgerm oil and/or cocoa butter.

Other ingredients may include essential oils of lemon, lavender, rose and/or chamomile to cleanse and freshen the skin. Witchhazel and juniper berry may be used to condition and tone skin. NAB spotted geranium extract from geranium plants may be included as an astringent and to reduce hemorrhaging. Antioxidants may include Vitamin C phoshpate or Vitamin E.

The invention may include the method of manufacturing the towelette, the towelette itself, and the use of the towelette on the pierced area of the person's skin. For example, the towelette product is prepared by mixing water and sea salt in a composition. The composition is applied to a fibrous sheet. The moistened fibrous sheet is then sealed in an individual foil wrapper or in another moisture-proof container.

As compared to large bottles of expensive piercing after care treatment, there is no waste or spillage and unlike a bottle, which is opened and shelved for long periods, the towelette remains in a hermetically sealed package until it is used and discarded. To facilitate the use of the towelette, a tattoo or piercing parlor may have a dispenser for self-serve and individual dispensing packaged towelettes one at a time.

Changes may be made in the above apparatus and process without departing from the scope of the invention herein. Thus, all matter contained in the above description or shown in the accompanying drawing shall be interpreted in an illustrative and not in a limiting sense. For example, advantageous results still could be achieved if steps of the disclosed techniques were performed in a different order and/or if components in the disclosed systems were combined in a different manner and/or replaced or supplemented by other components. Accordingly, other implementations are within the scope of the following claims.

I claim:

1. A body piercing towelette, comprising:
a composition comprising
water and
salt; and
a fibrous sheet pre-moistened with the composition.

2. The body piercing towelette of claim 1, wherein the water comprises deonized water.

3. The body piercing towelette of claim 1, wherein the water comprises filtered water.

4. The body piercing towelette of claim 3, wherein the filtered water comprises reverse osmosis filtered water.

5. The body piercing towelette of claim 1, wherein the water comprises sterilized water.

6. The body piercing towelette of claim 1, wherein the salt comprises sea salt.

7. The body piercing towelette of claim 6, wherein the sea salt comprises coral reef sea salt.

8. The body piercing towelette of claim 1, wherein the composition further comprises a preservative.

9. The body piercing towelette of claim 8, wherein the preservative includes lysozyme chloride.

10. The body piercing towelette of claim 1, wherein the composition further comprises an anti-inflammatory agent.

11. The body piercing towelette of claim 10, wherein the anti-inflammatory agent includes yucca glauca root extract.

12. The body piercing towelette of claim 1, wherein the composition further comprises an anti-irritant agent.

13. The body piercing towelette of claim 1, wherein the composition further comprises an anti-hemorrhaging agent.

14. The body piercing towelette of claim 13, wherein the anti-hemorrhaging agent includes yucca glauca root extract.

15. The body piercing towelette of claim 1, wherein the composition further comprises an antioxidant.

16. The body piercing towelette of claim 17, wherein the antioxidant comprises Vitamin C.

17. The body piercing towelette of claim 1, further comprising:
a sealed container to enclose the fibrous sheet.

18. The body piercing towelette of claim 1, wherein the sealed container comprises a moisture proof foil wrapper to enclose the fibrous sheet.

19. A method of producing a body piercing towelette, the method comprising:
mixing a composition of water and sea salt;
applying the composition to a fibrous sheet; and
enclosing the fibrous sheet in a sealed foil wrapper.

20. A method of treating a pierced area of a person's skin comprising:
removing a towelette impregnated with a mixture of sea salt and water from a sealed foil wrapper; and
swabbing the pierced area of the skin with the towelette.
